(19) United States
(12) Patent Application Publication
(10) Pub. No.: US 2005/0027102 A1
(43) Pub. Date: Feb. 3, 2005

(54) Use of molecularly imprinted polymers for stereo- and/or regioselective synthesis

(76) Inventors: Klaus Mosbach, Furulund (SE); Ian A. Nicholls, Lund (SE); Olof Ramstrom, Lund (SE)

Correspondence Address: MORGAN & FINNEGAN, L.L.P., Three World Financial Center, New York, NY, 10281-2101, US

(21) Appl. No.: 10/923,727

(22) Filed: Aug. 24, 2004

Related U.S. Application Data
(63) Continuation of application No. 09/645,500, filed on Aug. 25, 2000, now abandoned.
Application No. 09/645,500 is a continuation of application No. 08/992,861, filed on Aug. 6, 1997, now abandoned.
Application No. 08/992,861 is a continuation of application No. 08/464,832, filed on Jun. 27, 1995, now abandoned.

(30) Foreign Application Priority Data
Dec. 28, 1992 (SE) 9203913-0

(51) Int. Cl. C07H 1/00

(52) U.S. Cl. 530/333; 536/1.11

(57) ABSTRACT

Molecularly imprinted polymers can be utilized in stereo- and regio-selective synthesis. These systems can be utilized, e.g. for peptide synthesis, by selectively coordinating reactants at a predetermined preformed cavity. Further, such polymers may be used for the selective removal of one enantiomeric species from solution, allowing reaction to be directed to another species in bulk solution leading to stereoselective and/or regio-selective synthesis in the cavity of for instance peptides. Additionally, when utilized as regioselectively interacting protectioning matrices, these polymers can direct reaction to an alternate centre of a reacting substrate.

This invention refers to the use of molecularly imprinted polymers for stereo- and regioselective synthesis.

“Molecular imprinting” is the name given to a process for preparing polymers that are selective for a particular compound (the “print molecule”). The technique involves: 1) prearranging the print molecule and the monomers and allowing complementary interactions to develop; 2) polymerizing around the print molecule-monomer complex; and 3) removing the print molecule from the polymer by extraction (FIG. 1). Polymerization thus preserves the complementarity to the print molecule, and subsequently the polymer will selectively adsorb the print molecule. The print molecule binds more favourably to the extracted polymer than to structural analogues. The technique has also been referred to as “host-guest” polymerization and “template” polymerization. Preparation of the selective polymers is easy, involving only simple, well-known laboratory techniques.

Usually, one of two fundamentally different approaches has been followed in applying molecular imprinting: 1) the print molecule has been covalently, but reversibly bound; or 2) the initial interactions between monomers and the print molecule have been non-covalent. The covalent approach involves, in contrast to the non-covalent approach, the cumbersome covalent bond formation between the print molecule and the monomer (polymer).

Most organic reactions are carried out in free solution, one exception being catalysts immobilized on a solid matrix. Another example is the formation or sequencing of macromolecules such as peptides or polynucleotides following the so-called Merrifield approach where synthetic reactions are taking place on solid phase.

One disadvantage in using conventional solution chemistry is: Since several reactive groups in e.g. condensation reactions can often be involved, a great number of isomers, may they be regio- or stereoisomers, can be formed. To avoid the latter complications, various strategies of protecting such functional groups are being used.

The event of molecular imprinting involving, as described above, the formation of specific imprints (e.g. regio- and/or stereoselective) allows in principle synthetic reactions to take place in the cavities formed. The cavities will thus direct the synthesis in the desirable direction. In addition, it is possible that the surrounding polymer matrix will “take over” the function of the protecting groups. An additional fringe benefit of the approach is the fact that, because the cavities are specific, crude samples can be used, whereby the desired reaction products in a polymer matrix can subsequently be specifically isolated.

Furthermore, the repeated use of the polymer matrix is of great potential advantage and isolation of the products are made easier.

It is now a well established technique to mix an imprint molecule with monomers and crosslinkers followed by their polymerization around the imprint molecule and extraction of the latter. The monomers of often different functionalities interact during imprinting as well as subsequent recognition by non-covalent interactions such as ion-pair formation, dipolar electrostatic interactions, hydrogen bonding, charge transfer interactions and metal coordination (2, 3, 4). Alternatively, covalent interactions between imprint molecule and polymerizable monomers can be used (1). The most widely used monomers include various acrylates, heteroaromatics and vinylbenzenes. Such imprints can, according to the present invention, be used for chemical synthesis.

The additional aspect of using such imprints for catalysis, i.e. involving turnover, is an area of potential interest which however requires a number of prerequisites to be fulfilled such as the correct positioning of the catalyst or penetration of the catalyst through the polymer as well as easy dissociation of the formed products.

### SHORT DESCRIPTION OF THE DRAWINGS

FIG. 1 shows the principle of molecular imprinting.

FIG. 2 shows the use of molecularly imprinted polymers for the selective removal of one enantiomeric or regio-isomeric species from solution, while reaction takes place with the antipode or regio-isomer in bulk solution.

FIG. 3 shows molecularly imprinted polymers used as interacting protecting matrix.

FIG. 4 shows the selective benzoylation of a carbohydrate derivative,

FIG. 5 shows the use of molecularly imprinted polymers for directed synthesis.

FIG. 6 shows the site-specific coupling of N-protected L-tryptophanyl cloride to DL-phenylalanine methyl ester.

FIG. 7 shows the chemoselective synthesis of N-protected amino acids.

FIG. 8 shows the combined use of the direction and protection strategies.

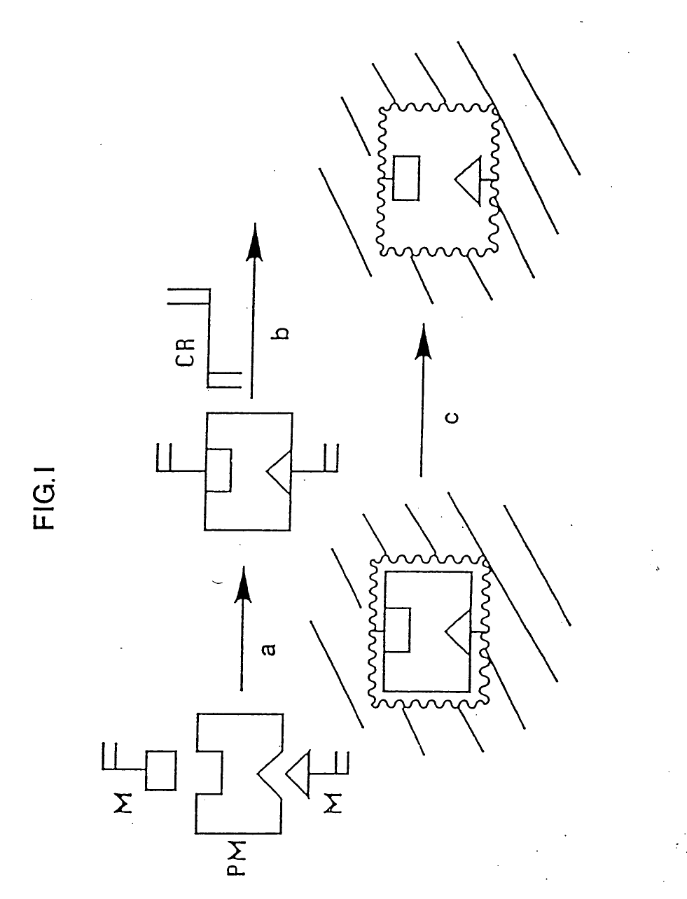

Further to the above, FIG. 1 shows the development of complementary interactions between the print molecule and the monomers (a); polymerization (b); removal of the print molecule from the polymer (c). M=monomers, PM=print molecule, CR=crosslinker.

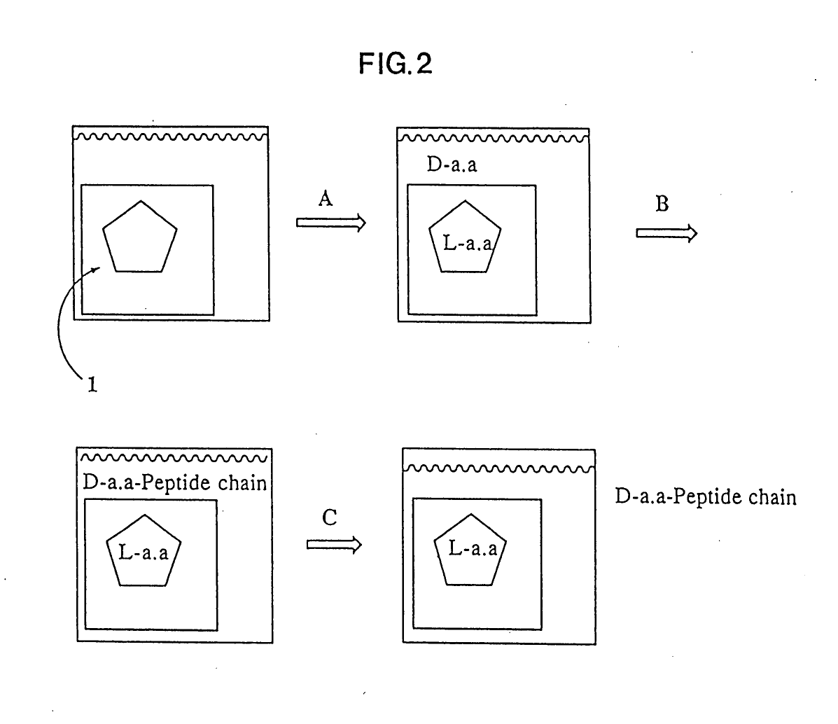

One aspect of the invention describes the use of molecularly imprinted polymers for the selective removal of one enantiomeric or regio-isomeric species from solution, while reaction takes place with the enantiopode or regio-isomer in bulk solution, e.g. in the synthesis of peptides, oligosaccarides and oligonucleotides (5). This can be achieved by the preparation of suitable imprinted polymer, careful manipulation of reaction stoichiometry and selection of suitable condensation reagents. This aspect is outlined in FIG. 2, in which 1 symbolizes a molecularly imprinted polymer against, in this case, an L-enantiomer of an amino acid or amino acid derivative. The polymer in solution is incubated with the racemic mixture of the amino acid or amino acid derivative during step A leading to the selective enrichment, by non-covalent interactions, of the L-enantiomer in the polymer and the D-enantiomer in solution. A suitable coupling reagent together with the adequately protected amino acid or peptide chain are introduced in step B. After coupling with the D-enantiomer in solution, the peptide chain is isolated by filtration in step C, whereas the L-enantiomer remains primarily in the polymer throughout the whole process.

Example. 1 below describes the enantioselective synthesis of a dipeptide, N-acetyl-D-tryptophanyl-L-phenylalanin methyl ester, utilizing a polymer imprinted against N-acetyl-L-tryptophan and, after incubation with the racemic mixture thereof, subsequent condensation with L-phenylalanine methyl ester in the bulk solution.

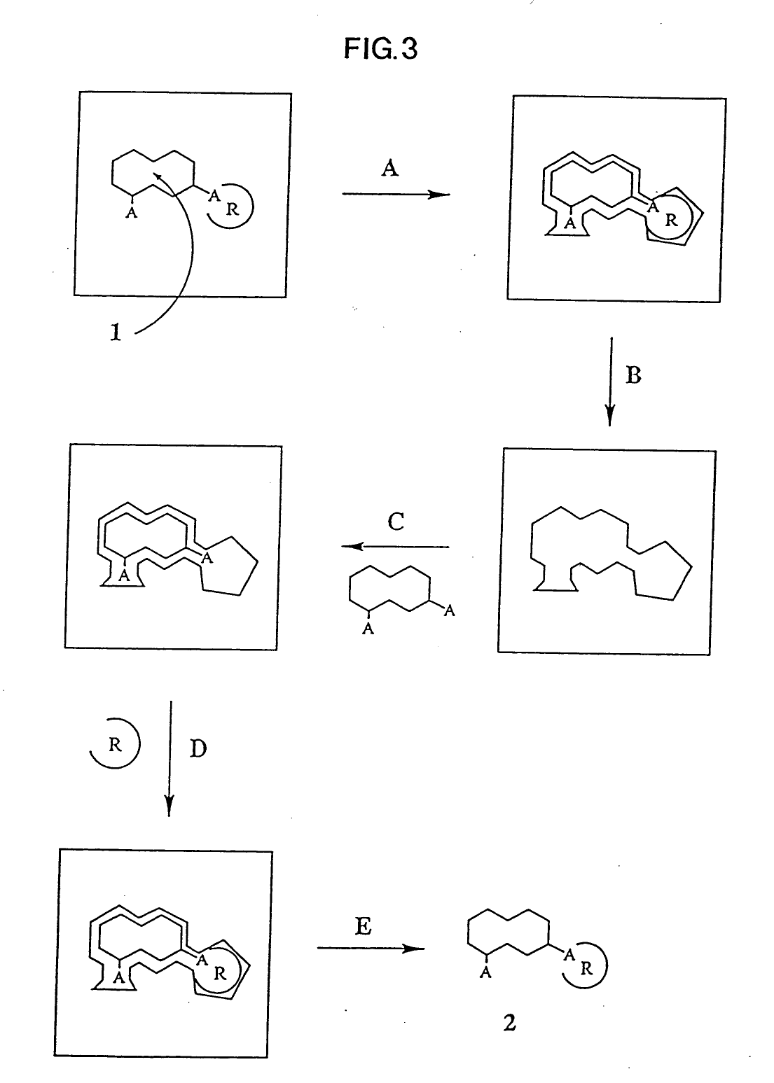

Another aspect of the invention covers the use of such imprinted polymers to act as interacting protecting matrices, capable of regio-selectively preventing a particular reaction at a specific site of a molecule containing several potentially reactive sites. This aspect is outlined in FIG. 3. A carefully selected substrate (1) incorporating two reactive sites (A), selectively protected in one position by a protecting group (R), is imprinted by non-covalent interactions in the designed polymer during step A. Exhaustive extraction of the polymer during step B leaves the polymer with complementary imprints of 1. Following incubation of the non-protected substrate analogue in step C, addition of a protecting group reagent (R or R′) in step D leads to site-specific reaction with the free functional group (A). Finally, the product is isolated by extraction of the polymer in step E.

It is conceived that, for example, in the case of peptide synthesis, the following groups can be substituted by interaction with the imprinted polymer matrix, may it be by complementary binding or by the surrounding matrix per se:

| Protection of | |
|---|---|
| functional group | Protecting group |
| Amino | tert-butyloxycarbonyl (BOC) |
| | 9-fluorenylmethyloxycarbonyl (Fmoc) |
| | benzyloxycarbonyl (Cbz) |
| | biphenylisopropyloxycarbonyl (Bpoc) |
| Carboxyl | methyl |
| | tert-butyl |
| | benzyl |
| Hydroxyl | tert-butyl |
| | benzyl |
| Thiol | benzyl |
| | acetimidomethyl (Acm) |
| Guanidino | nitro |
| | tosyl |
| | adamantyloxycarboflyl (Adoc) |
| Imidazol | benzyloxymethyl (BOM) |

The same concept should be valid also for synthesis of other compounds such as carbohydrates, nucleotides, etc.

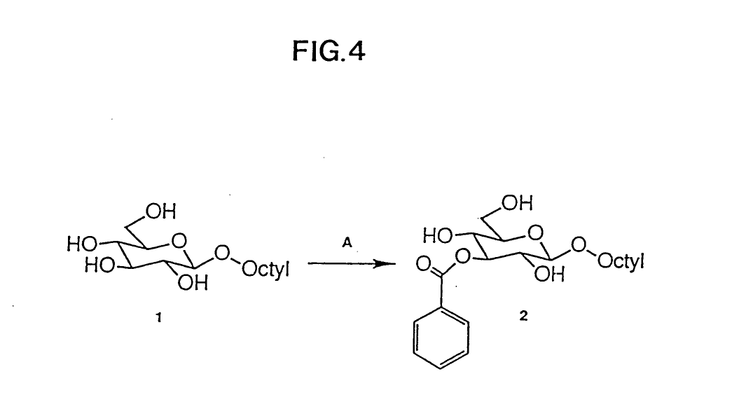

One example of this aspect is e.g. the selective benzoylation of a carbohydrate derivative utilizing selective protection of hydroxy groups by binding of the carbohydrate derivative to an imprinted polymer matrix, as indicated in FIG. 4. In this example, a polymer is imprinted against octyl-3-O-benzoyl-β-D-glucopyranoside (2) resulting in sites complementary to the above compound with the surrounding polymer matrix serving as protecting “agent” at the 2-O-, 4-O-, and 6-O-positions. Incubation of these polymers with octyl-β-D-glucopyranoside (1) and subsequent reaction with a suitable benzoylating agent (A) renders preferably the 3-O-benzoyl-derivative in favour of the 2-O-, 4-O-, or 6-O-derivatives.

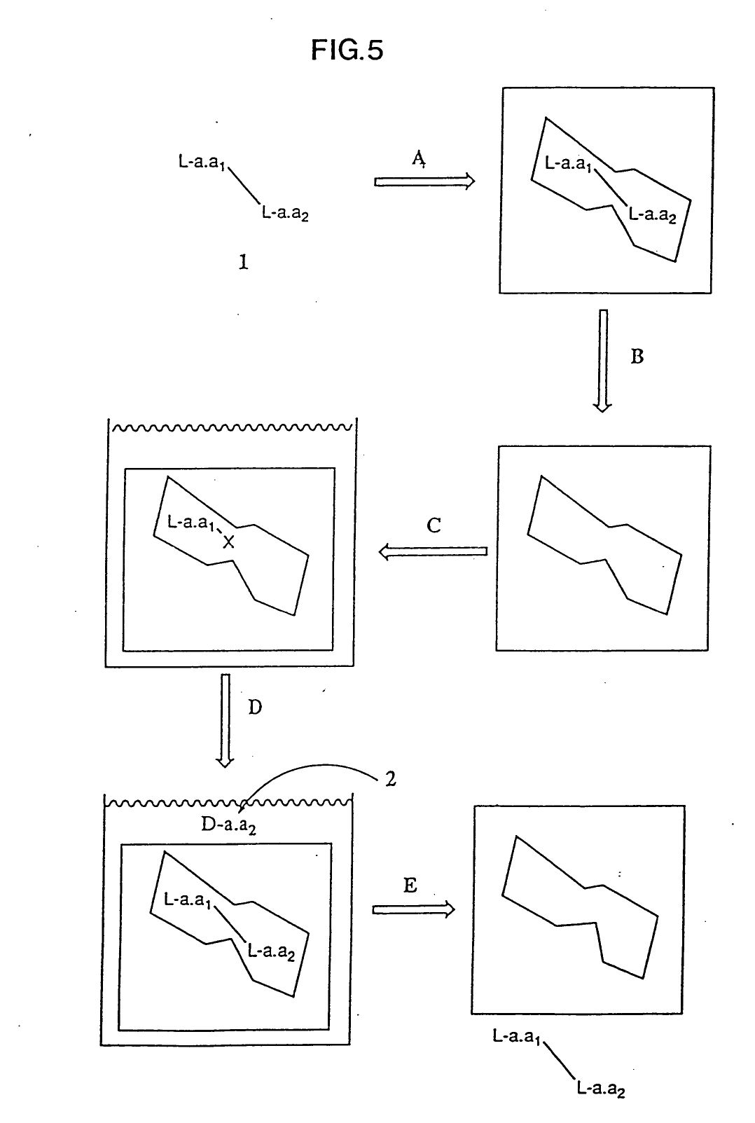

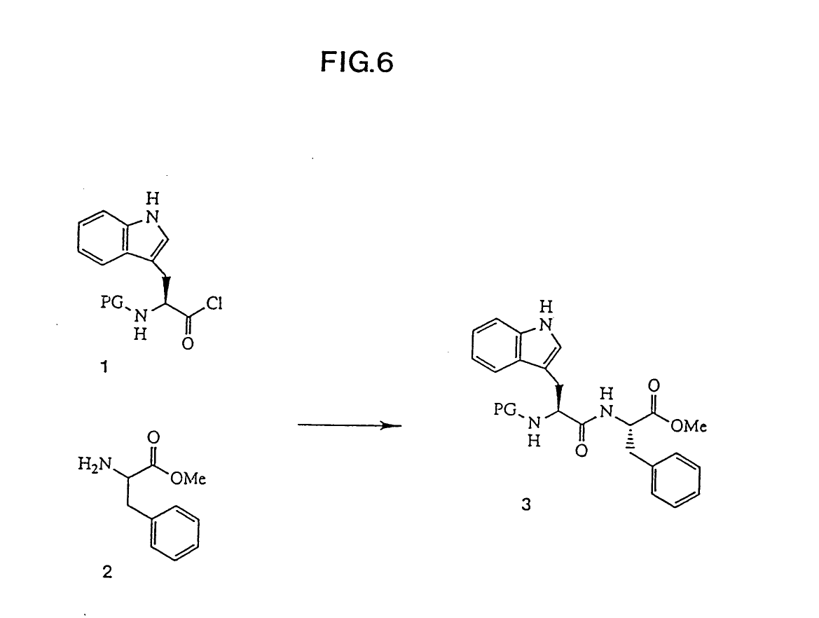

A third aspect of the present invention covers the use of such imprinted polymers for directed synthesis such as enantioselective synthesis of peptides, whereby condensation of amino acid (derivatives) is allowed to take place in the preformed recognition cavity. This aspect is described in FIG. 5. A carefully selected template or imprint species, in this case a suitably protected dipeptide (1), is imprinted by non-covalent interactions during step A. Extraction of the template in step B, results in a polymer matrix containing complementary recognition sites for 1. An activated form of amino acid 1 (L-a.a₁-X) is incubated in the polymeric sites during step C and addition of a racemic mixture of the second amino acid (DL-a.a₂) during step D leads to specific condensation in the cavity of the L-enantiomer forming the dipeptide corresponding to the template species (1). The resulting product is finally isolated by extraction in step E. An example of this strategy is e.g. the site-specific coupling of N-protected L-tryptophanyl chloride to DL-phenylalanine methyl ester (FIG. 6) where PG represents a protecting group such as e.g. a benzyloxycarbonyl (Cbz) group. In this example, a polymer is imprinted against the N-protected dipeptide N-PG-L-tryptophanyl-L-phenylalanine methyl ester (3) leading to the formation of sites complementary in shape and functionality to this imprint molecule. Incubation of the polymer with N-PG-tryptophanyl chloride (1) followed by addition of DL-phenylalanine methyl ester (2) effects the preferential synthesis of the imprint species (3), thus minimizing formation of N-PG-L--tryptophanyl-D-phenylalanine methyl ester.

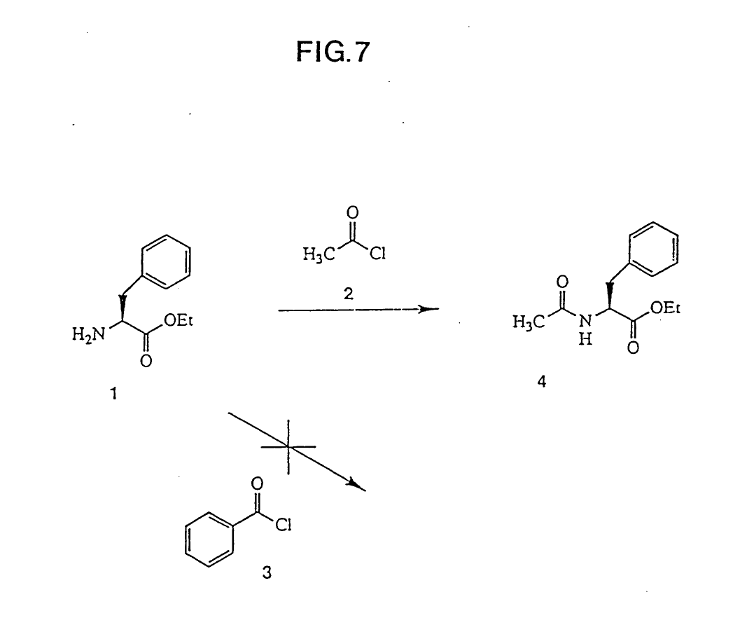

Another example of this aspect is the chemoselective synthesis of N-protected amino acids as outlined in FIG. 7. A polymer is imprinted against N-acetyl-L-phenylalanine ethyl ester (4) resulting in recognition sites complementary to this particular N-acetylated amino acid ester. Incubation of the polymer matrix with L-phenylalanine ethyl ester (1) and subsequent addition of either acetyl chloride (2) or benzoyl chloride (3) leads to preferential formation of the imprinted molecule (4), whereas the formation of the benzoylated derivative is inhibited. In a mixture of the acylating reagents, polymer-assisted formation of a high yield of N-acetyl-L--phenylalanine ethyl ester is obtained as compared to N-benzoyl-L-phenylalanine ethyl ester.

An additional example along these lines is the practically useful regio-selective synthesis of triglycerides from glycerol and various fatty acids. In this case, molecularly imprinted polymers can be used to direct the specific condensation of certain fatty acids with the glycerol moiety in order to obtain a required triacylglyceride.

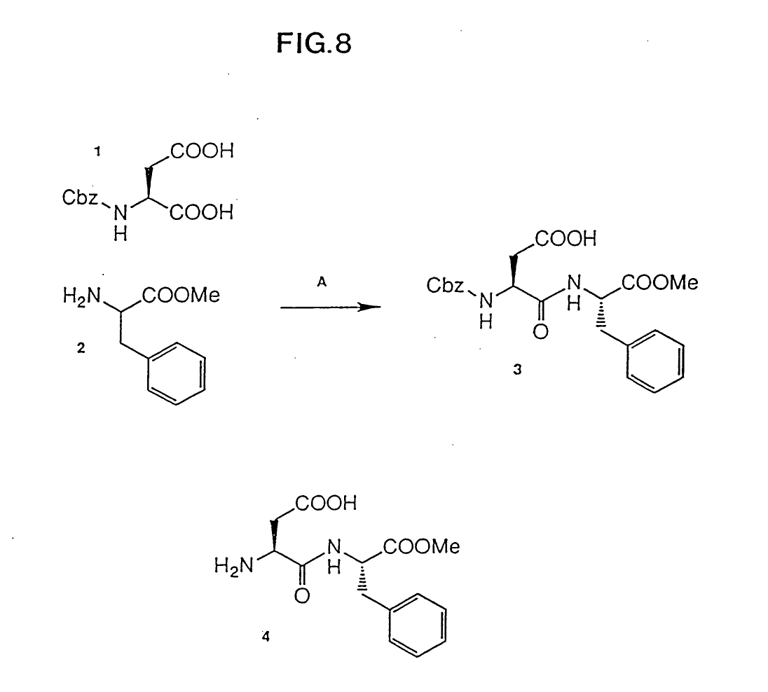

An example of employing molecularly imprinted polymers for applications combining both the direction and protection strategies is outlined in FIG. 8. For instance, these polymers can be imprinted against derivatives of molecules originally containing two or more identical functional groups, e.g. the dipeptide N-benzyloxycarbonyl--L-aspartyl-L-phenylalanine methyl ester (3). The β-carboxy group of this template species is unprotected and the carboxy group in the α-position of the aspartic acid residue is coupled to the phenylalanine residue. The resulting polymer leaves specific enantio- and regioselective interaction sites for the template molecule serving as a protecting matrix for the β-carboxy group. Incubation of the polymer with N-benzyloxycarbonyl-L-aspartic acid (1) and subsequent addition of DL-phenylalanine methyl ester (2) under suitable coupling conditions such as with reagents (A) renders preferably the α-dipeptide, whereas the β-isomer cannot be formed. Furthermore, as the imprint was prepared against the L-form of the second amino acid, preferential coupling with the L-form will occur in the cavity. Subsequent removal of the Cbz-group leads to the formation of the industrially important sweetening agent α-aspartame (4).

Another example is found in the area of conversion of antibiotics. For instance, in cavities obtained from cephalosporin C, selective cleavage of the side-chain leading to the useful 7-aminocephalosporanic, 7-ACA, can take place, alternatively similar imprints can be used for directed synthesis of semisynthetic cephalosporins from 7-ACA.

Another example utilizing both the surrounding polymer matrix as substitute for protecting groups, especially hydroxyl groups, and directing the synthesis is to be found in the syntheses of carbohydrates such as disaccharides. In one case imprinting of the disacharide 4-O-(β-D--galactopyranosyl)-β-D-2-deoxy-2-(N-acetylamino)-glucopyranose is followed by extraction. Subsequent condensation in the cavities of D-galactose and N-acetyl-D-glucosamine leads to the original imprint molecule. Analogously the important compound metyl-3-O-(β-D-galactopyranosyl)--β-D-glucopyranoside can be synthesized in a similar fashion. The condensation could be carried out following the Fischer reaction using solvents saturated with gaseous HCl or by utilizing one activated monosaccharide obtained by bromination at the anomeric carbon (6).

### EXAMPLE 1

In a typical experiment, a molecularly imprinted polymer was prepared against N-acetyl-L-tryptophan. Racemic N-acetyl-tryptophan (10 mg/ml in dry dimethylformamide) was incubated overnight at 4° C. in the presence of the imprinted polymer (500 mg) in a total volume of 2 ml, made up with tetrahydrofuran. After cooling to 0° C., L-phenylalanine metyl ester (1 eq.) and 1-hydroxybenzotriazole (1.1 eq) were added, followed by N,N′-dicyclohexylcarbodiimide (1.1 eq.). The reaction mixture was allowed to stand for 24 h, at room temperature, then filtered and the residue washed successively with portions of tetrahydrofuran and methanol/acetic acid. The filtrate was concentrated to dryness in vacuo and the residue partitioned between ethyl acetate and saturated sodium bicarbonate. The organic phase was successively washed with aqueous citric acid, saturated sodium bicarbonate and water, then dried and concentrated in vacuo. The crude products were purified by preparative thin layer chromatography, isolated and analysed by nuclear magnetic resonance (NMR). 36% diastereomeric excess of N-acetyl-D-tryptophanyl-L-phenylalanine methyl ester over N-acetyl-L--tryptophanyl-L-phenylalanine methyl ester was obtained.

References

1. Ekberg, B., Mosbach, K. Molecular Imprinting: a Technique for Producing Specific Separation Materials, Trends Biotechnol., 7, 92-96, 1989.
2. O'Shannessy, D., Ekberg, B., Andersson, L. I., Mosbach, K. Recent Advances in the Preparation and Use of Molecularly Imprinted Polymers for Enantiomeric Resolution of Amino Acid Derivatives, J. Chromatogr., 470, 391-399, 1989.
3. Ramström, O., Andersson, L. I., Mosbach, K., Recognition Sites Incorporating both Pyridinyl and Carboxy Functionalities Prepared by Molecular Imprinting, J. Org. Chem., in press
4. Ramström, O., Nicholls, I. A., Mosbach, K., Synthetic Polymer peptide receptors: Stereoselective Recognition in Non-Covalent Molecularly Imprinted Polymers, Tetrahedron:Asymmetry, submitted for publication.
5. Nicholls, I. A., Ramström, O., Mosback, K. Enantioselective Mediation of Reactivity by Molecularly Imprinted Polymer Derived Antibody Combining Site Mimics, manuscript.
6. K. Nilsson, Trends in Biotechnology 1988, Vol 6, 256-264.

1. Use of a molecularly imprinted polymer for stereoselective and/or regioselective synthesis, whereby the polymer acts as a stereoselectively and/or regioselectively interacting matrix, capable of interacting with at least one reactive species and directing a reaction to a reactive site of said species.

2-9. (Canceled)

10. A method of using a molecularly imprinted polymer, comprising the steps of:
providing a molecularly imprinted polymer adapted for stereoselective and/or regioselective synthesis,
using said molecularly imprinted polymer as a stereoselective and/or regeioselectively binding matrix,
directing by said polymer of a stereoselective and/or regioselective reaction.

11. The method of claim 10, wherein said molecularly imprinted polymer is adapted to eliminate the need for at least one protecting group on a reactive species.

12. The method of claim 10, wherein said molecularly imprinted polymer is produced by imprinting a print molecule and monomers wherein the print molecule was covalently, but reversibly, bound to the monomers.

13. The method of claim 10, wherein the step of providing a molecularly imprinted polymer comprises selecting a molecularly imprinted polymer resulting from molecular imprinting by imprinting a print molecule and monomers wherein the initial interactions between the monomers and the print molecule were non-covalent.

14. A method for chemical synthesis, comprising the steps of:
providing a molecularly imprinted polymer which is imprinted for a reactive chemical species,
providing a solution having said chemical species, said solution being in contact with said molecularly imprinted polymer,
interacting said imprinted polymer with said chemical species,
reacting said chemical species with a reactant to produce a reaction product.

15. The method of claim 14, wherein said interaction is stereoselective and/or regioselective.

16. The method of claim 14, wherein said reaction product is a peptide or polypeptide.

17. The method of claim 14, wherein said reaction product is a disaccharide, oligosaccharide or polysaccharide.

18. The method of claim 14, wherein said reaction product is a dinucleotide, oligonucleotide or polynucleotide.
